(19) United States
(12) Patent Application Publication
(10) Pub. No.: US 2007/0197975 A1
(43) Pub. Date: Aug. 23, 2007

(54) Dose Metering Mechanism with Sequence Control

(76) Inventors: Stefan Burren, Bremgarten (CH); Ulrich Moser, Heimiswil (CH); Christian Schrul, Burgdorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/677,823

(22) Filed: Feb. 22, 2007

Related U.S. Application Data
(63) Continuation of application No. PCT/CH05/00470, filed on Aug. 12, 2005.

(30) Foreign Application Priority Data
Aug. 25, 2004 (DE) 10 2004 041 151.4

(51) Int. Cl. A61M 5/00

(52) U.S. Cl. 604/207

(57) ABSTRACT

A dosing device for the dosed administration of an injectable product from an injection device, the dosing device including an outer sleeve and a sliding sleeve which can be displaced in relation to the outer sleeve, wherein the sliding sleeve includes at least one flexible element including a first stop generally adjacent to a counter stop associated with the outer sleeve whereby the dosing device can define a relative displacement between the outer sleeve and the sliding sleeve.

### CROSS-REFERENCE TO RELATED APPLICATION(S)

This application is a continuation of International Application No. PCT/CH2005/000470, filed on Aug. 12, 2005, which claims priority to German Application No. 10 2004 041 151.4, filed on Aug. 25, 2004, the contents of both of which are incorporated in their entirety herein.

### BACKGROUND

The present invention relates to devices for delivering, injecting, administering, dispensing or infusing substances, and to methods of making and using such devices. More particularly, it relates to a dose metering mechanism for use in administering measured doses of an injectable product or substance from an injection device and, more particularly, to a dose metering mechanism by which a sequence control can be achieved when dispensing a substance from an ampoule or other reservoir associated with, for example, an injection device, thereby making it easy to set a priming dose and a dose to be dispensed which is different from the priming dose.

Injection devices, including those known as injection pens, designed for administering doses of a medicinal substance or a medicament such as insulin or hormones, typically must initially be set to dispense a priming quantity during what is referred to as a priming operation. In such priming operations, typically air and any residual substances are removed from the injection cannula due to the priming operation by ejecting a specific low or small quantity of the product to be injected from the needle of the injection device without effecting an injection. A standard, larger dose of the substance to be injected can then be set and/or administered.

On a known injection pen, a rotatable dose metering sleeve is provided, which has different stops and is in the form of a stepped element. Different stroke lengths of a toothed bar or plunger rod can be set by a dose metering sleeve so that a user usually has to set at least two different doses, namely a small dose of the substance to be dispensed or moved for priming and then the dose to be administered, which will then be injected by the injection device.

Even in situations where a constant predefined quantity or dose of the substance to be administered has to be injected, it is still necessary for a user to set the priming quantity first, which may result in incorrect operation on the part of the user whereby the dose to be administered may be used for priming, for example, or even the priming dose may be injected.

### SUMMARY

One object of the present invention is to provide a dose metering mechanism for administering measured doses of an injectable product from an injection device which makes operation of an injection device user-friendly, including during priming.

In one embodiment, the present invention comprises a dosing device for the dosed administration of an injectable product from an injection device, the dosing device comprising an outer sleeve and a sliding sleeve which can be displaced in relation to the outer sleeve, wherein the sliding sleeve includes at least one flexible element comprising a first stop generally cooperative with a counter stop associated with the outer sleeve whereby the dosing device can define a relative displacement between the outer sleeve and the sliding sleeve.

In one embodiment, a dose metering mechanism in accordance with the present invention for administering measured doses of an injectable product from an injection device comprises an outer sleeve and a forwardly displaceable sleeve displaceable relative to the outer sleeve. For the purpose of the invention, the forwardly displaceable sleeve is provided with at least one displaceable and, in some preferred embodiments, flexible element with a first stop, which lies against a complementary stop of the outer sleeve and is able to restrict a relative movement between the outer sleeve and the forwardly displaceable sleeve. Providing such a displaceable and/or flexible element and incorporating a first stop means that, if a profile is provided in a plunger rod or toothed bar or another forwardly displaceable element which moves to cause a substance to be dispensed from an ampoule, for example by pushing a plunger, the flexible element or a locating piece disposed on it is able to locate or move into a recess of a toothed bar, for example, to move the first stop into a position in which the forwardly displaceable sleeve can be freely moved relative to the outer sleeve. As the displaceable or flexible element or the locating piece disposed on it is pushed, via a ramp or another profile of the toothed bar, relative to the forwardly displaceable sleeve, in other words is pushed radially outwardly, the first stop is moved into a position in which the relative movement between the outer sleeve and the forwardly displaceable sleeve is restricted due to a complementary stop on the outer sleeve being moved against the stop of the forwardly displaceable sleeve, so that the movement is halted on contact or abutment of the stops.

In some embodiments, the first stop may be disposed on the forwardly displaceable sleeve so that the forwardly displaceable sleeve can be moved only until the complementary stop of the outer sleeve lies against the first stop, thereby enabling any further movement of the forwardly displaceable sleeve to be restricted. Another option is wherein the first stop is not pushed into an abutting position by a ramp of a toothed bar but is disposed such that, after the movement of the forwardly displaceable sleeve relative to the outer sleeve, a block is achieved to prevent further movement. In the context of the present invention, a stop or block may be any structure or operation able to restrict or halt a relative movement of two objects or sleeves.

In some embodiments, when the forwardly displaceable sleeve, which may be connected to a knob to facilitate operation by the user, is pulled backwards as part of a pulling movement on the injection device, for example by extracting or moving the knob toward an end of the injection device opposite the dispensing end of the injection device, it can be pulled only until it lies against a complementary stop of the outer sleeve, due to the first stop. This means that the distance between the stop on the forwardly displaceable sleeve and the complementary stop of the outer sleeve determines the maximum possible distance of a pulling movement and, hence, the maximum quantity of a dose of a substance to be dispensed. In some preferred embodiments, a profile or recess is provided in the toothed bar at only a smaller partial distance so that the flexible element of the forwardly displaceable sleeve is already pushed outwards after a relatively short pulling movement, as a result of which only a small quantity is set and dispensed for a first discharging or dispensing operation, for example for priming. Consequently, a user no longer has to set the priming dose separately because the automatic restricting action of the dose metering mechanism proposed by the invention ensures that a user can not set a larger dose than the priming dose for a first dispensing operation defined by the distance of the stops. For subsequent dispensing operations with a view to injecting a dose, a longer recess may be provided in the toothed bar after the ramp in the toothed bar, so that larger quantities of a substance to be dispensed can be metered and set. Accordingly, the selection of a dose quantity to be dispensed initially is automatically controlled by the dose metering mechanism in accordance with the present invention, so that a user is able to effect a priming operation with a small dose and then set a dose for the injection, without having to set the priming dose itself. A user therefore no longer has an option for setting the priming dose, which means that incorrect manipulations or incorrect settings of an injection device can be largely prevented and a user can readily control the operating sequence when using the injection device.

In some embodiments, an outer sleeve in accordance with the present invention can be fitted around or, alternatively, inside the forwardly displaceable sleeve and is advantageously connected to the external face of the injection device or at least forms a part of it, while the forwardly displaceable sleeve is advantageously connected to a setting element, such as a knob, for example.

In some embodiments, it may be preferable to provide two or more flexible elements on the forwardly displaceable sleeve, and a flexible element may be provided in the form of an elastic arm, for example, which is disposed in a cut-out or orifice of the forwardly displaceable sleeve. In the initial state, such flexible element (or elements) is advantageously pushed in the direction of a toothed bar or plunger rod, which is guided inside the forwardly displaceable sleeve, for example. A locating piece is provided on the forwardly displaceable sleeve, for example on the arm, and, this being case, advantageously points or extends in the direction of the toothed bar or plunger rod, in other words inwardly if using an approximately cylindrically shaped forwardly displaceable sleeve, for example.

In some embodiments, the first stop or a portion thereof may point or extend both inwardly and outwardly if using an approximately cylindrically shaped forwardly displaceable sleeve. In some embodiments, the stop may be provided in the form of a lug or cam, for example, thereby enabling the cam to move into abutment with a co-operating complementary stop associated with the outer sleeve to permit a relative axial movement of the forwardly displaceable sleeve slidingly mounted or carried in the outer sleeve, but only until the stops hit one another and, thus, automatically set a priming dose.

In some preferred embodiments, at least a second stop may be associated with or on the forwardly displaceable sleeve, for example in the form of a projecting lug or a cam, in which case the second stop is able to restrict a relative movement between the outer sleeve and forwardly displaceable sleeve if the forwardly displaceable sleeve is not restricted by the first stop. When a flexible element of the forwardly displaceable sleeve on which the first stop is disposed locates in a recess of a toothed bar due to the biasing action of the flexible element, thereby enabling a bigger dose than the priming dose to be set, it will be possible to specify how big a maximum dose set for an injection operation should be on the basis of the distance between the second stop and a complementary stop associated with or on the outer sleeve. In some preferred embodiments, the first and second stops may be offset from one another axially and may co-operate with the same or alternatively different stops associated with or on the outer sleeve.

In some embodiments, the present invention further relates to a dose metering mechanism of the type described above, with a toothed bar guided by the outer sleeve and/or forwardly displaceable sleeve and, in some preferred embodiments, inside the forwardly displaceable sleeve, and having at least one or, in some embodiments, two or more recesses which define a dispensing profile. If at least two recesses are provided one after the other along the axial length of the toothed bar or plunger rod, for example, these individual recesses may be separated from one another by a ramp or raised areas, so that a locating piece of the flexible element of the forwardly displaceable sleeve locating in the recesses can be pushed outwards away from the mid-axis of the toothed bar or plunger rod by such a ramp. In this case, the first stop provided on the forwardly displaceable sleeve and/or on the flexible element is pushed radially outwardly away from the toothed bar to restrict a pulling movement of the injection device in conjunction with a complementary stop of the outer sleeve to set or fix a priming dose. In principle, only a single recess may be provided on the toothed bar, in which the flexible element of the forwardly displaceable sleeve can locate after priming so that the first stop is not able to affect the setting of another dose.

In some embodiments, catch elements are provided on the outer sleeve and/or forwardly displaceable sleeve or on a guide piece connected to the forwardly displaceable sleeve. The catch elements may advantageously be mounted on elastic or resilient elements or arms, in which case the catch elements are designed to permit a relative movement of the toothed bar, in which the catch elements locate, in one direction and block it in another direction.

In some preferred embodiments, the catch elements are designed so that they locate in co-operating recesses or teeth of a toothed bar so that the toothed bar can be moved in the direction of a dispensing orifice of the injection device relative to the outer sleeve or guide piece or relative to the forwardly displaceable sleeve.

In some embodiments, the present invention comprises an injection device with a dose metering mechanism of the type described above.

In some embodiments, the present invention further relates to a method of priming or preparing the measured dispensing of an injectable product from an injection device, wherein a first fitting movement of the injection device is restricted after a first predefined distance by an abutment of a first stop of a forwardly displaceable sleeve on a complementary stop of an outer sleeve, and a first dose, which may be referred to as a priming dose, is dispensed for priming purposes by pushing a toothed bar or plunger rod, and wherein the restriction on the relative movement between the outer sleeve and forwardly displaceable sleeve due to the first stop of the forwardly displaceable sleeve is released during or after dispensing the priming dose.

### BRIEF DESCRIPTION OF THE DRAWINGS

FIGS. 1A-1E illustrate a dose metering mechanism in accordance with one embodiment of the present invention with a toothed bar in different operating states; and

FIGS. 2 and 3 provide cross-sectional views of a flexible element of a forwardly displaceable sleeve locating in a toothed bar.

### DETAILED DESCRIPTION

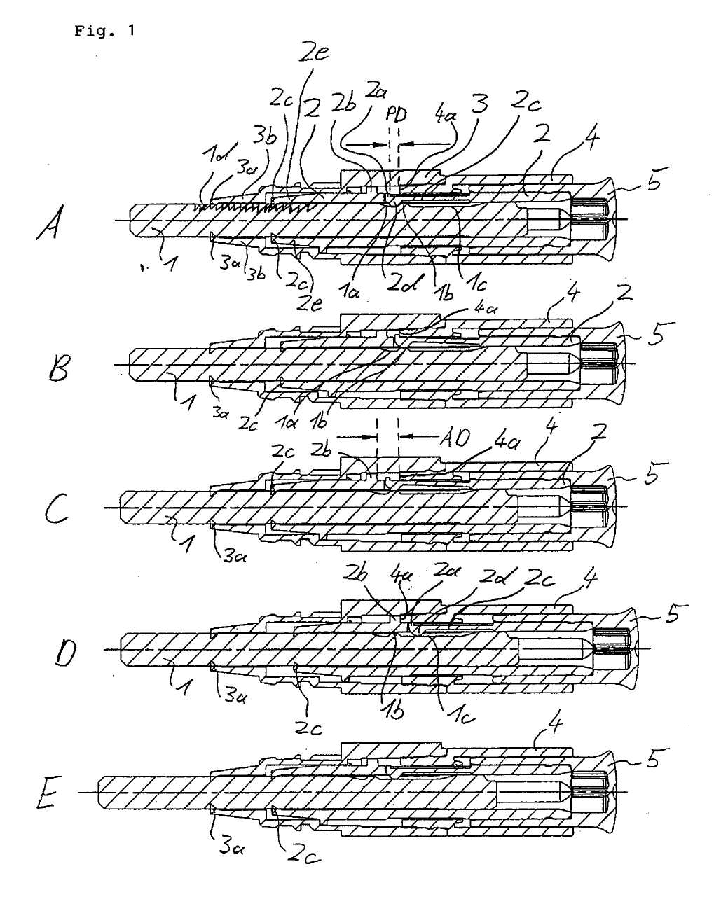

FIG. 1A illustrates one embodiment of a dose metering mechanism in accordance with the present invention. The mechanism may constitute the rear end of an injection device, for example, with a toothed bar 1 with schematically illustrated teeth 1d on the top face disposed inside a forwardly displaceable sleeve 2. The front end of the bar (on the left-hand side in FIG. 1A) may press against a stopper of an ampoule (not illustrated). The movement of the toothed bar 1 toward the left in FIG. 1A results in a dispensing operation of an injection device with which the dose metering mechanism is associated.

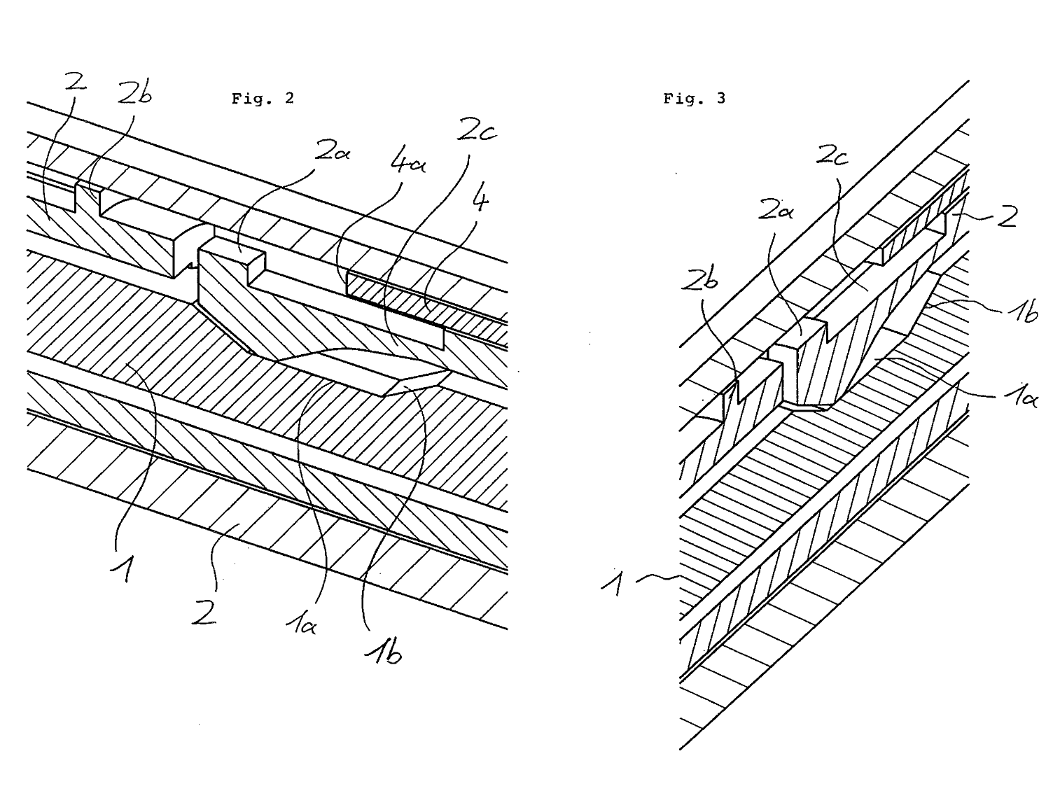

The forwardly displaceable sleeve 2 is disposed around the toothed bar 1. The sleeve has or carries a flexible arm, which may be referred to or thought of as a snap structure or snapper 2c, which, as also illustrated in FIGS. 2 and 3, is provided in an opening of the forwardly displaceable sleeve 2. The arm is able to move radially inwardly or outwardly, and the snap structure locates in a first recess 1a of the toothed bar 1 when in the position illustrated in FIG. 1A so that the flexible arm 2c is not biased in the position illustrated, which is of advantage because, in the case of a forwardly displaceable sleeve 2 made from plastic for example, the tension of a pre-biased snapper 2c might diminish or ease with time.

At the front end illustrated on the left-hand side in FIG. 1A, the forwardly displaceable sleeve 2 has elastic elements, such as resilient arms 2e, for example, the front end of which is provided with inwardly pointing catch elements 2c which can locate in recesses or the toothing 1d of the toothed bar 1. Thus, as illustrated in FIG. 1A, the toothed bar 1 can be pushed relative to the forwardly displaceable sleeve 2 in the direction of the dispensing opening of an injection device, toward the left in FIG. 1A, and any movement in the opposite direction is blocked or prevented by the catch elements 2c locating in recesses or teeth 1d on the external face of the toothed bar 1. The forwardly displaceable sleeve 2 is fixedly connected to a knob 5 serving as a setting element.

Provided around the forwardly displaceable sleeve 2 is an outer sleeve 4 with a stationary stop 4a, which can not be displaced by operating the knob 5. The outer sleeve 4 is connected to a guide piece 3, which, like the front part of the forwardly displaceable sleeve 2, has elastic arms 3b with catch elements 3a which are able to locate in the toothed bar 1 as described above and permit a relative movement in one direction.

The toothed bar 1 has elongated recesses 1a and 1c extending longitudinally, which are separated from one another by a ramp 1b. In the position illustrated, the flexible arm 2c of the forwardly displaceable sleeve locates in the first recess 1a of the toothed bar 1 so that a first stop 2a provided on the external face of the flexible arm 2c does not project radially beyond the circumference of the forwardly displaceable sleeve 2.

FIG. 1B illustrates the state of the dose metering mechanism after a user has pulled the dose metering mechanism, by pulling on the knob 5, towards the right side of FIG. 1B. The forwardly displaceable sleeve 2 connected to the knob 5 is moved relative to the outer sleeve 4 and the toothed bar 1 is retained by the catch elements 3a in the pulling direction, as a result of which the locating piece 2d provided on the flexible arm 2c migrates or moves along the ramp 1b of the toothed bar 1 and is thus pushed or moved radially outwardly. Thus, the lug 2a provided on the external face of the flexible arm 2c serving as a stop is pushed radially outwardly and lies against the stop 4a of the outer sleeve 4 at the end of the pulling distance PD (indicated in FIG. 1A). As a result, the pulling movement relative to the outer sleeve 4 effected by pulling on the knob 5 is restricted. The distance PD between the first stop 2a of the forwardly displaceable sleeve 2 and the stationary stop 4a of the outer sleeve 4 therefore fixes the maximum length of the pulling movement and hence the maximum quantity of the priming dose. During the pulling movement, a user pulls the knob 5 out of the outer sleeve 4, during which the toothed bar 1 is held stationary due to the catch elements 3a provided on the guide piece 3 fixedly connected to the outer sleeve 4, so that the toothed bar 1 is unable to move relative to the guide piece 3 and the outer sleeve 4 during the pulling operation. The catch elements 2d on the elastic arms 2e of the forwardly displaceable sleeve 2 thus migrate towards the rear along the toothed bar profile 1d in the axial direction of the toothed bar 1.

By pushing on the knob 5, it is pushed back into the outer sleeve 4 in readiness for dispensing or priming, as illustrated in FIG. 1C. As a result, the catch elements 2c provided on the front end of the forwardly displaceable sleeve 2 locate in the toothed bar 1 and transmit the pressure exerted by the forwardly displaceable sleeve 2 connected to the knob 5 to the toothed bar 1 causing it to be pushed towards the left-hand side of FIG. 1C, so that it pushes against a stopper of a typical ampoule (not illustrated), thus causing the priming dose to be dispensed. As this takes place, the forwardly displaceable sleeve 2 is not moved relative to the toothed bar. The catch elements 3a migrate along the toothed profile 1d of the toothed bar 1.

After priming or after dispensing the priming dose fixed on the basis of the distance PD, the dose metering mechanism can be pulled again to set or select a specific dose for an injection, as illustrated in FIG. 1D. The forwardly displaceable sleeve 2 is moved toward the rear again by pulling out the knob 5 relative to the toothed bar 1 retained by the catch elements 3a of the guide piece 3. The locating piece 2d is thus pushed away from the ramp 1b of the toothed bar 1 in the direction of the second recess 1c of the toothed bar 1 and pushed in the radial direction inwardly into the second recess 1c by the elastic force of the flexible arm 2c so that the lug 2a, constituting the first stop, is moved away from the stationary stop 4a of the outer sleeve 4. This enables the setting or selection of a bigger dose than that defined by the distance PD.

During the pulling motion, the forwardly displaceable sleeve 2 can be pushed until the cam 2b provided on the external face of the forwardly displaceable sleeve 2 as a second stop lies against the stationary stop 4a of the outer sleeve 4 and thus restricts the pulling movement. The distance AD (indicated in FIG. 1C) between the second stop 2b of the forwardly displaceable sleeve 2 and the stop 4a of the outer sleeve 4 therefore defines the maximum length of a pulling movement and thus a dispensing dose for an injection. The pulling movement for setting the dispensing dose is therefore no longer restricted by the first stop 2a. As described above in connection with the pulling movement used to effect priming, the catch elements 3a of the guide piece 3 retain the toothed bar 1, while the catch elements 2c of the forwardly displaceable sleeve 2 migrate or move along the toothed bar 1.

A set dose can then be dispensed by depressing the knob 5 and sliding the forwardly displaceable sleeve 2, as illustrated in FIG. 1E. As this takes place, the toothed bar 1 is pushed by the catch elements 2c of the forwardly displaceable sleeve 2 together with the forwardly displaceable sleeve 2 in the direction of a dispensing orifice of the injection device, causing the catch elements 3a of the guide piece 3 to migrate along the toothed bar 1.

Accordingly, a sequence and/or control of sequential steps is obtained by moving the first stop 2a on the flexible arm 2c of the forwardly displaceable sleeve 2 in and out by the profile in the toothed bar 1, the profile being defined by the recesses 1a and 1c and the ramp 1b. Different sequences and/or sequences of different doses can be set, predetermined or selected on the basis of the length and/or disposition of different recesses or profiles on, in or associated with the toothed bar 1. It would also be possible to provide other stops, for example, in addition to the stationary stop 4a of the outer sleeve 4, in co-operating recesses and/or cut-outs of the outer sleeve 4, or other ramps and recesses in the profile of the toothed bar 1.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dosing device for the dosed administration of an injectable substance from an injection device, the dosing device comprising an outer sleeve and a sliding sleeve displaceable relative to the outer sleeve, wherein the sliding sleeve comprises a first stop generally cooperative with a counter stop associated with the outer sleeve whereby the dosing device defines a relative displacement between the outer sleeve and the sliding sleeve.

2. The dosing device according to claim 1, wherein sliding sleeve further comprises a flexible element comprising the first stop.

3. An injection device for the dosed administration of an injectable substance, the injection device comprising a dosing mechanism comprising an outer sleeve and a sliding sleeve displaceable relative to the outer sleeve, wherein the sliding sleeve comprises a first stop generally cooperative with a counter stop associated with the outer sleeve whereby the dosing mechanism defines a relative displacement between the outer sleeve and the sliding sleeve.

4. A dose metering mechanism for administering measured doses of an injectable product from an injection device, the dose metering mechanism comprising an outer sleeve, a displaceable sleeve displaceable relative to the outer sleeve and carrying a displaceable element comprising a first stop able to abut a complementary stop associated with the outer sleeve, wherein the stops restrict a relative movement between the outer sleeve and the forwardly displaceable sleeve.

5. The dose metering mechanism according to claim 4, wherein the displaceable element is flexible.

6. The dose metering mechanism as claimed in claim 4, further comprising a setting element fixedly connected to the displaceable sleeve.

7. The dose metering mechanism as claimed in claim 6, wherein the setting element is a knob.

8. The dose metering mechanism as claimed in claim 4, wherein the displaceable element is an arm associated with the displaceable sleeve.

9. The dose metering mechanism as claimed in claim 8, wherein the arm is elastically mounted in a cut-out of the displaceable sleeve.

10. The dose metering mechanism as claimed in claim 4, wherein the first stop is one of a cam or lug carried by the flexible element.

11. The dose metering mechanism as claimed in claim 4, wherein the displaceable sleeve is displaceably mounted in the outer sleeve.

12. The dose metering mechanism as claimed in claim 4, wherein a second stop is provided on the displaceable sleeve.

13. The dose metering mechanism as claimed in claim 4, further comprising a toothed bar having at least one recess.

14. The dose metering mechanism as claimed in claim 13, wherein the toothed bar has at least two axially separated recesses.

15. The dose metering mechanism as claimed in claim 14, wherein the recesses are separated by a ramp.

16. The dose metering mechanism as claimed in claim 13, wherein the displaceable sleeve, the outer sleeve and a guide piece connected to the outer sleeve comprise catch elements cooperative with the teeth of the toothed bar.

17. The dose metering mechanism according to claim 16, wherein the catch elements are carried by resilient arms.

18. An injection device comprising a dose metering mechanism comprising a toothed bar, an outer sleeve and a sliding sleeve displaceable relative to the outer sleeve, wherein the sliding sleeve comprises a first stop generally cooperative with a counter stop associated with the outer sleeve whereby the dosing device defines a relative displacement between the outer sleeve and the sliding sleeve.

19. The injection device according to claim 18, wherein the displaceable sleeve, the outer sleeve and a guide piece connected to the outer sleeve comprise catch elements cooperative with the teeth of the toothed bar

20. A method of preparing for the dispensing of an injectable product in measured doses from an injection device, comprising the steps of:
exerting a force in one direction on a portion of the injection device thereby moving the portion, the movement of the portion being restricted after a predefined distance due to an abutment of a first stop associated a displaceable sleeve and a complementary stop associated with another sleeve,
dispensing a priming dose determined by the predefined distance by exerting a force on the portion in another direction thereby moving one of a toothed bar or plunger rod; and
releasing the restriction during or after dispensing the priming dose.

21. The method according to claim 20, further comprising the step of again exerting said force in one direction to select an injection dose.
